(19) United States
(12) Patent Application Publication
(10) Pub. No.: US 2025/0244307 A1
(43) Pub. Date: Jul. 31, 2025

(54) RECONSTRUCTION OF THE COTTON FIBER LENGTH DISTRIBUTION FROM A FIBROGRAM

(71) Applicant: Texas Tech University System, Lubbock, TX (US)

(72) Inventors: Christopher Turner, Lubbock, TX (US); Md Abu Sayeed, Lubbock, TX (US); Eric Hequet, Lubbock, TX (US)

(73) Assignee: Texas Tech University System, Lubbock, TX (US)

(21) Appl. No.: 18/854,789

(22) PCT Filed: Apr. 4, 2023
(86) PCT No.: PCT/US2023/017450

§ 371 Date: Oct. 7, 2024

Related U.S. Application Data
(60) Provisional application No. 63/327,623, filed on Apr. 5, 2022.

(51) Int. Cl. G01N 33/36; G01B 11/02

(52) U.S. Cl. CPC G01N 33/365; G01B 11/02

(57) ABSTRACT

Embodiments of the present disclosure pertain to computer-implemented methods of reconstructing a fiber length distribution of a fiber from its fibrogram by receiving the fibrogram; determining an end of the fibrogram; applying a windowed curve-fitting procedure to smoothen the fibrogram curvel and estimating the fiber length distribution of the fiber from the smoothened fibrogram curve. The methods may also include one or more steps of reconstructing an initial and missing portion of the fibrogram; assessing fiber quality based on the estimated fiber length distribution; adjusting one or more fiber-related conditions based on the estimated fiber length distribution; and repeating the method after the adjustment. Additional embodiments pertain to computing devices for reconstructing a fiber length distribution of a fiber from its fibrogram.

### CROSS-REFERENCE TO RELATED APPLICATIONS

This application claims priority to U.S. Provisional Patent Application No. 63/327,623, filed on Apr. 5, 2023. The entirety of the aforementioned application is incorporated herein by reference.

### STATEMENT REGARDING FEDERALLY SPONSORED RESEARCH

This invention was made with government support under A20-187, awarded by the U.S. Department of Agriculture. The government has certain rights in the invention.

### BACKGROUND

Current methods of assessing fiber length distribution have numerous limitations. Embodiments of the present disclosure aim to address the aforementioned limitations.

### SUMMARY

In some embodiments, the present disclosure pertains to computer-implemented methods of reconstructing a fiber length distribution of a fiber from its fibrogram. In some embodiments, the methods of the present disclosure include the steps of receiving the fibrogram, where the fibrogram includes a curve representing the number of fibers present at a given length from the base of the fiber; determining an end of the fibrogram; applying a windowed curve-fitting procedure to smoothen the fibrogram curve; and estimating the fiber length distribution of the fiber from the smoothened fibrogram curve. In some embodiments, the methods of the present disclosure also include one or more steps of reconstructing an initial and missing portion of the fibrogram; assessing fiber quality based on the estimated fiber length distribution; adjusting one or more fiber-related conditions based on the estimated fiber length distribution; and repeating the method after the adjustment.

Additional embodiments of the present disclosure pertain to computing devices for reconstructing a fiber length distribution of a fiber from a fibrogram of the fiber. In some embodiments, the computing device includes one or more computer readable storage mediums that have a program code embodied therewith. In some embodiments, the program code includes programming instructions for: receiving the fibrogram; determining an end of the fibrogram; applying a windowed curve-fitting procedure to smoothen the fibrogram curve; and estimating the fiber length distribution of the fiber from the smoothened fibrogram curve. In some embodiments, the program code also includes one or more programming instructions for reconstructing an initial and missing portion of the fibrogram; assessing fiber quality based on the estimated fiber length distribution; instructing the adjustment of one or more fiber-related conditions based on the estimated fiber length distribution; and instructing the repetition of the programming instructions after the implementation of the adjustment step.

### DESCRIPTION OF THE DRAWINGS

FIG. 1A illustrates a computer-implemented method for reconstructing a fiber length distribution of a fiber from its fibrogram.

FIG. 1B provides an illustration of a computing device for reconstructing a fiber length distribution of a fiber from a fibrogram.

FIG. 2 shows an image of a fiber beard.

FIG. 3 shows a graph of a sample fibrogram.

FIG. 4 shows the fibrogram of a monolength fiber sample. N is the number of fibers and L is the length.

FIG. 5 shows a tangent line to the fibrogram at length x with a y-intercept at Y(x).

FIG. 6 shows a fiber beard with an example of a nep as well as a trash particle.

FIGS. 7A-7C provide synthetic distributions that are used to test the algorithm. FIG. 7A shows a Gaussian mixture. FIG. 7B shows a Weibull mixture representing an immature/weak cotton. FIG. 7C shows a Weibull mixture representing a mature/strong cotton.

FIGS. 8A-8B show results of applying the reconstruction algorithm on the fibrogram from the Gaussian mixture distribution as Y(x) (FIG. 8A) and n(x) (FIG. 8B) graphs. In each graph. the “True” line is the true value, and the “Cubic Fit” line is obtained from the algorithm.

FIGS. 9A-9B show results of applying the reconstruction algorithm on the fibrogram from the distribution representing an immature/weak cotton. Shown are Y(x) (FIG. 9A) and n(x) (FIG. 9B) graphs. In each graph, the “True” line is the true value, and the “Cubic Fit” line is obtained from the algorithm.

FIGS. 10A-10B show results of applying the reconstruction algorithm on the fibrogram on the distribution representing a mature/strong cotton. Shown are Y(x) (FIG. 9A) and n(x) (FIG. 9B) graphs. In each graph, the “True” line is the true value, and the “Cubic Fit” line is obtained from the algorithm.

FIG. 11 shows average R2 values over 60 iterations of leave-one-out cross-validation (LOOCV) for each predictor-response combination.

FIG. 12 shows MSE values over 60 iterations of LOOCV for each predictor-response combination.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore. the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in

A driving force behind the textile and cotton industries is controlling for measurement of cotton fiber length distribution. By managing this factor, higher quality of both cotton (i) production and (ii) end-use transformation can more readily be assured. Taking a look at the two main devices on the market that currently measure cotton fiber distribution, namely High Volume Instrument (HVI) and Advanced Fiber Information System (AFIS), provides some insights on their shortcomings.

Conventional HVI is the most common fiber quality evaluation system, even though it fails to capture certain fiber qualities that contribute to improved yarn production, most relevant of which is fiber length variation in a sample (i.e., distribution). AFIS has the advantage of individualizing fibers and isolating dust interference from the sample. However, AFIS is prone to fiber breakage. Commercial methods such as HVI and AFIS are lacking as their respective parameters (e.g., upper half mean length and uniformity index) only provide limited distribution information.

As such, a need exists for improved methods of estimating fiber length distribution. Numerous embodiments of the present disclosure aim to address the aforementioned limitations.

### Methods of Reconstructing Fiber Length Distributions

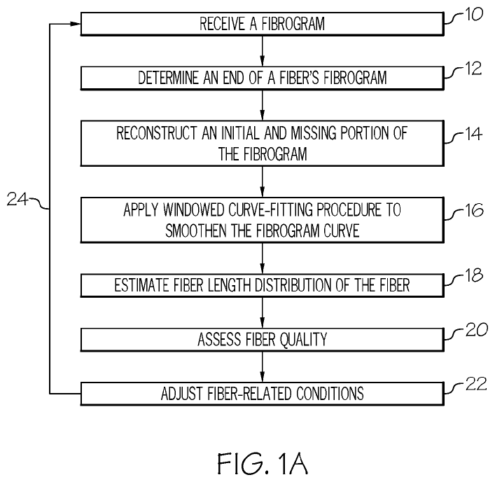

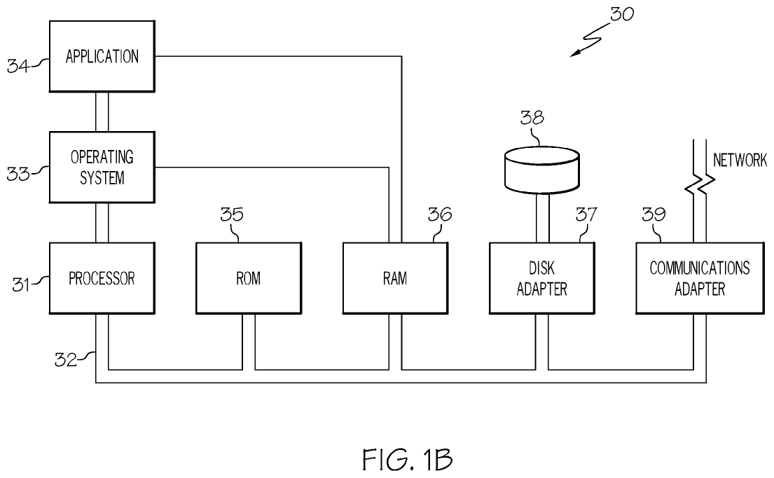

In some embodiments, the present disclosure pertains to computer-implemented methods of reconstructing a fiber length distribution of a fiber from its fibrogram. In some embodiments illustrated in FIG. 1A, the methods of the present disclosure include the steps of receiving the fibrogram, where the fibrogram includes a curve representing the number of fibers present at a given length from the base of the fiber (step 10); determining an end of the fibrogram (step 12); applying a windowed curve-fitting procedure to smoothen the fibrogram curve (step 16); and estimating the fiber length distribution of the fiber from the smoothened fibrogram curve (step 18). In some embodiments, the methods of the present disclosure also include a step of reconstructing an initial and missing portion of the fibrogram (step 14). In some embodiments, the methods of the present disclosure also include one or more steps of assessing fiber quality based on the estimated fiber length distribution (step 20); adjusting one or more fiber-related conditions based on the estimated fiber length distribution (step 22); and repeating the steps after the adjustment (step 24). As set forth in more detail herein, the methods of the present disclosure can have numerous embodiments.

### Fibrograms

The methods of the present disclosure may utilize various types of fibrograms. For instance, in some embodiments, the fibrograms include fibrograms constructed through the utilization of a High Volume Instrument (HVI).

In some embodiments, the methods of the present disclosure also include a step of constructing a fiber's fibrogram. For instance, in some embodiments, the fibrogram is constructed through the utilization of an HVI.

### Determining an End of the Fibrogram

In some embodiments, an end of a fibrogram represents a length value of the fibrogram that is zero. In some embodiments, an end of a fibrogram represents a first discrete difference of the fibrogram that is greater than or equal to zero. In some embodiments, an end of a fibrogram represents a point at which the longest fibers have been scanned after which the remaining data is assumed to be zero.

Various methods may be utilized to determine an end of a fibrogram. For instance, in some embodiments, a polynomial curve fit procedure is utilized to determine the end of the fibrogram.

### Reconstructing an Initial and Missing Portion of the Fibrogram

In some embodiments, the methods of the present disclosure include an additional step of reconstructing an initial and missing portion of a fiber's fibrogram. In some embodiments, the reconstruction occurs through the utilization of a convex function.

### Applying a Windowed Curve-Fitting Procedure to Smoothen a Curve

Various methods may be utilized to apply a windowed curve-fitting procedure to smoothen a curve of a fibrogram. For instance, in some embodiments, the windowed curve-fitting procedure includes polynomial smoothing through a sliding window method. In some embodiments, the sliding window method utilizes differentiable and parametric functions. In some embodiments, when fit to underlying data in a given window, the function is convex within a domain of the window.

In some embodiments, the windowed curve-fitting procedure also removes slightly concave portions from a fibrogram curve. In some embodiments, the windowed curve-fitting procedure also estimates derivatives of fibrogram equations.

### Estimating an Underlying Fiber Length Distribution

Various methods may be utilized to estimate an underlying fiber length distribution. For instance, in some embodiments, the estimating of the underlying fiber length distribution includes estimating a cumulative distribution function and a probability function.

In some embodiments, the estimating of the underlying fiber length distribution is based on a given window size and a parametric curve. In some embodiments, the window size is selected such that the underlying data within the window is generally convex. In some embodiments, the curve for the smoothing process has a function that is differentiable and parametric, and when fit to the underlying data in the window, the function is convex within the domain of the window. In some embodiments, the curve is generally smooth within the window. In some embodiments, the curve is capable of providing a good approximation within any window along the fibrogram. In some embodiments, the window size is about 6.35 mm.

The estimating of the underlying fiber length distribution can occur at various distance intervals. For instance, in some embodiments, the estimating is simultaneous with the applying of the windowed curve-fitting procedure.

### Assessment of Fiber Quality

In some embodiments, the methods of the present disclosure also include a step of assessing fiber quality based on the estimated fiber length distribution. For instance, in some embodiments, the methods of the present disclosure include a step of designating a grade or a number to the fiber based on the estimated fiber length distribution.

### Adjusting Fiber-Related Conditions

In some embodiments, the methods of the present disclosure also include a step of adjusting one or more fiber-related conditions based on the estimated fiber length distribution. In some embodiments, the adjustment step includes instructing a user to adjust the one or more fiber-related conditions based on the estimated fiber length distribution. In some embodiments, the adjustment step occurs manually by a user. In some embodiments, the adjustment step occurs manually by a user without any computer-implemented instructions.

The methods of the present disclosure may be utilized to adjust various fiber-related conditions. For instance, in some embodiments, the one or more fiber-related conditions to be adjusted include, without limitation, fiber growth conditions, fiber storage conditions, fiber milling conditions, fiber transport conditions, fiber breeding conditions, or combinations thereof.

In some embodiments, the one or more fiber-related conditions include one or more fiber growth conditions. In some embodiments, the one or more fiber growth conditions include, without limitation, herbicide levels, irrigation conditions, fertilizer levels, growth temperature, or combinations thereof.

In some embodiments, the methods of the present disclosure also include a step of repeating the reconstruction of the fiber length distribution after the implementation of the adjustment step. For instance, in some embodiments, the methods of the present disclosure may be utilized to continuously assess fiber length distribution and fiber quality after making various adjustments of fiber-related conditions.

### Fibers

The methods of the present disclosure may be utilized to assess the fiber length distributions of various fibers. For instance, in some embodiments, the fibers include, without limitation, textile fibers, cotton fibers, hemp fibers, natural bast fibers, flax fibers, jute fibers, kenaf fibers, milkweed fibers, ramie fibers, artificial fibers, or combinations thereof.

In some embodiments, the fibers include cotton fibers. In some embodiments, the cotton fibers include, without limitation, upland cottons, pima cottons, viscose cottons, or combinations thereof. In some embodiments, the fibers include cotton fiber beards.

The fibers of the present disclosure may be in various forms. For instance, in some embodiments, the fibers of the present disclosure are in the form of an aggregated fiber. In some embodiments, the aggregated fiber is in the form of a fiber beard, a fiber bundle, a yarn, or combinations thereof.

In some embodiments, the aggregated fiber is in the form of a fiber bundle. In some embodiments, the aggregated fiber is in the form of a fiber board. In some embodiments, the aggregated fiber is in the form of a fiber yarn. In some embodiments, the fiber yarn includes ring-spun yarns, air-jet-spun yarns, or combinations thereof.

### Computing Devices for Reconstructing Fiber Length Distributions

Additional embodiments of the present disclosure pertain to computing devices for reconstructing a fiber length distribution of a fiber from its fibrogram. In some embodiments, the computing device includes one or more computer readable storage mediums that have a program code embodied therewith. In some embodiments, the program code includes programming instructions for: receiving the fibrogram, where the fibrogram includes a curve representing the number of fibers present at a given length from the base of the fiber; determining an end of the fibrogram; applying a windowed curve-fitting procedure to smoothen the fibrogram curve; and estimating the fiber length distribution of the fiber from the smoothened fibrogram curve.

In some embodiments, the program code also includes programming instructions for reconstructing an initial and missing portion of the fibrogram. In some embodiments, the reconstructing occurs through the utilization of a convex function.

In some embodiments. the computer program code also includes programming instructions for assessing fiber quality based on the estimated fiber length distribution. In some embodiments, the program code also includes programming instructions for instructing the adjustment of one or more fiber-related conditions based on the estimated fiber length distribution. In some embodiments, the one or more fiber-related conditions include. without limitation, fiber growth conditions, fiber storage conditions, fiber milling conditions, fiber transport conditions, fiber breeding conditions, or combinations thereof. In some embodiments, the one or more fiber-related conditions include one or more fiber growth conditions, such as herbicide levels, irrigation conditions, fertilizer levels, growth temperature, or combinations thereof. In some embodiments, the program code also includes programming instructions for instructing the repetition of the programming instructions after the implementation of the adjustment step.

The computing devices of the present disclosure can have numerous embodiments. For instance, in some embodiments, the program code utilizes a polynomial curve fit procedure to determine an end of a fibrogram.

In some embodiments, the programming instructions for the windowed curve-fitting procedure includes polynomial smoothing through a sliding window method. In some embodiments, the sliding window method utilizes differentiable and parametric functions. In some embodiments, when fit to underlying data in a given window, the function is convex within a domain of the window.

In some embodiments, the programming instructions for the windowed curve-fitting procedure also removes slightly concave portions from the fibrogram curve. In some embodiments, the programming instructions for the windowed curve-fitting procedure also estimates derivatives of fibrogram equations.

In some embodiments, the programming instructions for estimating of the underlying fiber length distribution is simultaneous with programming instructions for the applying of the windowed curve-fitting procedure. In some embodiments, the programming instructions for estimating of the underlying fiber length distribution includes instructions for estimating a cumulative distribution function and a probability function.

In some embodiments, the programming instructions for estimating of the underlying fiber length distribution is based on a given window size and a parametric curve. In some embodiments, the window size is selected such that the underlying data within the window is generally convex. In some embodiments, the curve for the smoothing process has a function that is differentiable and parametric, and when fit to the underlying data in the window, the function is convex within the domain of the window. In some embodiments, the curve is generally smooth within the window. In some embodiments, the curve is capable of providing a good approximation within any window along the fibrogram.

The computing devices of the present disclosure can include various types of computer readable storage mediums. For instance, in some embodiments, the computer readable storage mediums can be a tangible device that can retain and store instructions for use by an instruction execution device. In some embodiments, the computer readable storage medium may include, without limitation, an electronic storage device, a magnetic storage device, an optical storage device, an electromagnetic storage device, a semiconductor storage device, or combinations thereof. A non-exhaustive list of more specific examples of suitable computer readable storage medium includes, without limitation, a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, or combinations thereof.

A computer readable storage medium, as used herein, is not to be construed as being transitory signals per se. Such transitory signals may be represented by radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer readable program instructions described herein can be downloaded to respective computing/processing devices from a computer readable storage medium or to an external computer or external storage device via a network, such as the Internet, a local area network, a wide area network and/or a wireless network. In some embodiments, the network may include copper transmission cables, optical transmission fibers, wireless transmission, routers, firewalls, switches, gateway computers and/or edge servers. In some embodiments, a network adapter card or network interface in each computing/processing device receives computer readable program instructions from the network and forwards the computer readable program instructions for storage in a computer readable storage medium within the respective computing/processing device.

In some embodiments, computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. In some embodiments, the computer readable program instructions may execute entirely on the user's computer, partly on the user's computer, as a stand-alone software package. partly on the user's computer and partly on a remote computer or entirely on the remote computer or server. In the latter scenario, the remote computer may be connected in some embodiments to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry in order to perform aspects of the present disclosure.

Embodiments of the present disclosure for reconstructing cotton fiber length distribution from a fibrogram as discussed herein may be implemented using a computing device illustrated in FIG. 1B. Referring now to FIG. 1B, FIG. 1B illustrates an embodiment of the present disclosure of the hardware configuration of a computing device 30 which is representative of a hardware environment for practicing various embodiments of the present disclosure.

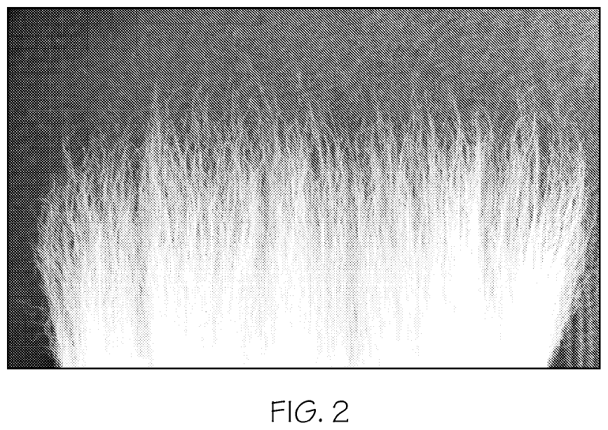

Computing device 30 has a processor 31 connected to various other components by system bus 32. An operating system 33 runs on processor 31 and provides control and coordinates the functions of the various components of FIG. 1B. An application 34 in accordance with the principles of the present disclosure runs in conjunction with operating system 33 and provides calls to operating system 33, where the calls implement the various functions or services to be performed by application 34. Application 34 may include, for example, a program for reconstructing cotton fiber length distribution from a fibrogram as discussed in the present disclosure, such as in connection with FIGS. 2-6, 7A-7C, 8A-8B, 9A-9B, 10A-10B and 11-12.

Referring again to FIG. 1B, read-only memory (“ROM”) 35 is connected to system bus 32 and includes a basic input/output system (“BIOS”) that controls certain basic functions of computing device 30. Random access memory (“RAM”) 36 and disk adapter 37 are also connected to system bus 32. It should be noted that software components including operating system 33 and application 34 may be loaded into RAM 36, which may be computing device's 30 main memory for execution. Disk adapter 37 may be an integrated drive electronics (“IDE”) adapter that communicates with a disk unit 38 (e.g., a disk drive). It is noted that the program for reconstructing cotton fiber length distribution from a fibrogram, as discussed in the present disclosure, such as in connection with FIGS. 2-6, 7A-7C, 8A-8B, 9A-9B, 10A-10B and 11-12, may reside in disk unit 38 or in application 34.

Computing device 30 may further include a communications adapter 39 connected to bus 32. Communications adapter 39 interconnects bus 32 with an outside network (e.g., wide area network) to communicate with other devices.

Aspects of the present invention are described herein with reference to flowchart illustrations and/or block diagrams of methods, apparatus (systems), and computing devices according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computing devices according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Applications and Advantages

The methods and computing devices of the present disclosure can provide numerous advantages and applications. In particular, traditional fibrograms encounter (a) imperfections caused by trash particles; (b) lapses in scanning near the comb: and (c) the erroneous collection of data beyond the longest fiber scan. In some embodiments, the methods and computing devices of the present disclosure address these issues by (1) identifying and limiting collection of data no further than the longest fiber; and (2) reconstructing the initial gap near the comb based on a convex function. Thus, in some embodiments, the methods and computing devices of the present disclosure provide significant commercial value in resolving an identifiable problem endemic within fiber production industries, such as the cotton production industry.

In some embodiments, a strength of the methods and the computing devices of the present disclosure is their accuracy in reconstruction and potential superiority to AFIS and HVI regarding fiber (e.g., yarn) quality prediction. These fiber (e.g., cotton) quality predictions are a core consideration of spinning mills when purchasing fibers (e.g., cotton lint) and introducing end products to the market.

Moreover, the methods and computing devices of the present disclosure do not require many parts or complex equipment. As such, the methods and computer programs of the present disclosure can be introduced inexpensively and without significant regulatory oversight.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### EXAMPLE 1

### Reconstruction of the Cotton Fiber Length Distribution from a High Volume Instrument VR fibrogram

In the cotton industry, the evaluation of cotton fiber quality is of vital importance. Not only docs fiber quality determine the selling price of cotton, but it also assists garment manufacturers in configuring their equipment. One of the most important factors in cotton fiber quality is fiber length for which there are several often-used properties such as mean length (ML), upper half mean length (UHML), and short fiber content (SFC). To measure these properties, the cotton industry employs a variety of tools and instruments some of which base their length measurements on the fibrogram concept.

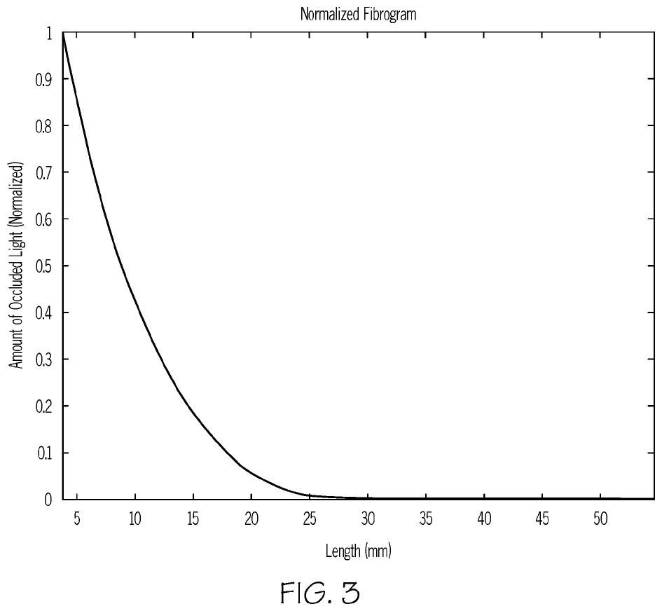

The fibrogram is a type of length-frequency curve that describes the distribution of fibers in a prepared fiber beard, which is a sample of paralleled fibers as shown in FIG. 2. By definition, the fibrogram represents the number of fibers present at a given length from the base of the fiber beard (i.e., the comb). A seminal work on fibrogram theory is attributed to K. L. Hertel (Hertel, 1940). In his work, Hertel describes a fiber beard prepared from a sliver and analyzed using a fibrograph. The fibrograph is a device that uses photovoltaic cells to measure the amount of light occluded by the fibers at various lengths along the fiber beard. The amount of light occluded is considered an approximation of the number of fibers at that length. The resulting curve of the amount of occluded light versus length is the fibrogram. The amount of occluded light is typically normalized such that the fibrogram ranges from zero to one as shown in FIG. 3.

Since 1940, other instruments have been developed to generate fibrograms in a similar manner. One prevalent example is the USTER High Volume Instrument (HVI). Rather than preparing a fiber beard from a sliver, the HVI uses a fibrosampler to generate a fiber beard. Like the fibrograph, the HVI also uses occluded light versus displacement to produce a fibrogram curve.

In this Example, Applicant presents an algorithm that can reconstruct a fiber length distribution from a fibrogram regardless of the method in which the fibrogram was constructed. However, Applicant does not assume that the fiber beard is prepared in such a way that all fibers have an equal probability of being selected from a cotton sample regardless of fiber length, as is the case with the fibrosampler. The algorithm is based on an extension of fibrogram theory and employs windowed curve fitting techniques to reconstruct the length distribution of the fibers present in the fiber beard as well as the cumulative distribution function. In the following sections, Applicant reviews the fibrogram theory, discusses the details of the new algorithm, and demonstrates its effectiveness on synthetic data as well as yarn quality prediction using real cotton samples.

### EXAMPLE 1.1

### Review of Fibrogram Theory

As mentioned supra, the seminal work on fibrogram theory was put forth by K. L. Hertel in 1940 (Hertel, 1940). In generating the fiber beard from a sliver, one of the key assumptions Hertel makes is that “the fiber is to be selected at random and every point on every fiber is equally probable.” Later, Chu and Riley put forth that this assumption by Hertel translates into two key points (Chu and Riley, 1997): (1) A sampled fiber is held at a random point along its length. (2) The probability of sampling a particular fiber is proportional to its length.

However, Chu and Riley altered these assumptions as they were establishing the fundamental principles of a fibrogram based on a fiber beard prepared by a fibrosampler. “The mathematic techniques developed by Hertel may be modified for the assumption that every fiber has an equal probability of being caught by the comb needles regardless of its length. The holding point along each sampled fiber length is assumed to be random. Thus, for a monolength fiber sample, the shape of the fibrogram is triangular.” (Chu and Riley, 1997).

It is important to note that these new assumptions necessitate that Chu and Riley's theoretical formulation supersede Hertel's when dealing with fibrograms generated from samples prepared by an instrument with the same sampling assumptions as the fibrosampler.

Continuing with Chu and Riley's explanation, given a sample of length L and a total number of fibers N, the fibrogram R measured at a distance x can be written as equation 1.

$$R ( x ) = N ( L - x ) / L , \qquad ( 1 )$$

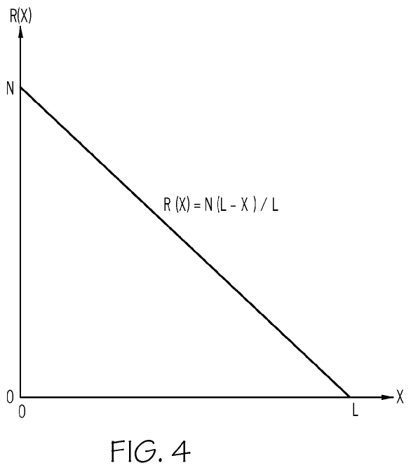

In equation 1, R is in units of the number of fibers. FIG. 4 shows a graphical representation of equation 1.

Expanding on (1), if instead of a mono length sample, a length frequency distribution of the fiber board is given, n(L), the equation for the fibrogram then becomes equation 2.

$$R ( x ) = {\int }_{χ}^{{L}_{m}} \frac{( L - x ) n ( L )}{L} dL , \qquad ( 2 )$$

In equation 2, Lm is the length of the longest fibers in the sample. Equation 3 is also applicable.

$$R ( 0 ) = {\int }_{0}^{{L}_{m}} \frac{( L - 0 ) n ( L )}{L} dL = {\int }_{0}^{{L}_{m}} n ( L ) dL = N . \qquad ( 3 )$$

From equation 2, one can calculate the first and second derivatives of R(x), as outlined in equations 4 and 5.

$${R}^{′} ( x ) = - {\int }_{x}^{{L}_{m}} \frac{n ( L )}{L} dL \qquad ( 4 )$$

$${R}^{″} ( x ) = \frac{n ( x )}{x} \qquad ( 5 )$$

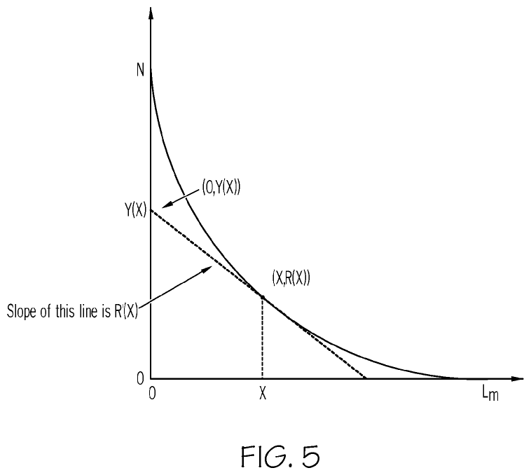

Furthermore, Chu and Riley describe a tangent line on the fibrogram at a distance x as shown in FIG. 5.

Using the coordinates of two points along the line, the equation of this line can be written as equation 6.

$$Y ( x ) = R ( x ) - {xR}^{′} ( x ) . \qquad ( 6 )$$

By plugging in equations 2 and 4 into equation 6 and solving, one can obtain equation 7.

$$Y ( x ) = {\int }_{x}^{{L}_{m}} n ( L ) dL . \qquad ( 7 )$$

In equation 7, Y(x) is a cumulative distribution function (CDF) i.e., Y(x)=P(X≥x). From equation 7, one can compute the distribution, n(x), as equation 8.

$$n ( x ) = - \frac{dY ( x )}{d x} . \qquad ( 8 )$$

Finally, equation 9 considers the integral I1 (0,Lm), which is defined as the area of the fibrogram, which is half of the total length of fibers in the fiber beard.

$$I 1 ( 0 , {L}_{m} ) = {\int }_{0}^{{L}_{m}} R ( x ) d x = \frac{1}{2} {\int }_{0}^{{L}_{m}} x n ( x ) dx , \qquad ( 9 )$$

Therefore, mean length, ML, is given as equation 10.

$$M L = 2 \times \frac{I 1 ( 0 , {L}_{m} )}{R ( 0 )} . \qquad ( 10 )$$

Chu and Riley go on to show how the standard deviation of the mean length can also be derived. However, that statistic is not commonly used by the industry.

The authors conclude by stating that the “length distribution, mean length, and standard deviation can be calculated from the fibrogram” (Chu and Riley, 1997), which is true from a theoretical point of view. However, reconstructing the length distribution from fibrograms acquired via some type of sensor system presents a set of problems that must be addressed. In the following section, we explain our method for reconstructing the length distribution while also considering some practical issues that arise in a fibrogram representation regardless of the sensing method.

The algorithm Applicant has developed attempts to apply Chu and Riley's theoretical formulation in a practical setting. It resolves several issues that may be encountered when using various sensing methods to estimate a fibrogram from a fiber beard. In the sections herein, Applicant describes the types of problems that may be encountered when acquiring a fibrogram from a typical fiber beard and how Applicant can overcome them.

### EXAMPLE 1.2

### Theory Versus Real World Application

One of the most important factors in reconstructing the distribution from fibrograms is the assumption of convexity in the theoretical fibrogram. According to equation 5, since the length distribution, n(x), is always positive, and only positive lengths are applicable (i.e., x>0), the second derivative of the fibrogram in equation 5 is always positive. Hence, equation 2 is a convex function for x>0.

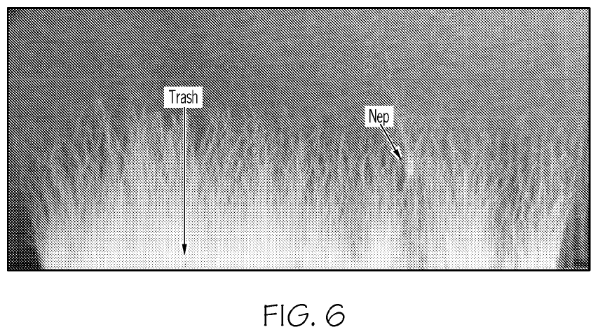

However, in practice, a fibrogram acquired from a fiber beard may exhibit areas of concavity due to a number of factors. First, imperfections in the fiber beard may be caused by small trash particles as well as neps, as shown in FIG. 6. In theory, as the beard is scanned from the base to the tips of the fibers, one would not expect the density of the beard to increase. However, these imperfections may cause a perceived increase in fiber beard density, which would manifest as concavity in the fibrogram. Another major source of concavity could be the comb to which the fibers in the beard are attached. Typically, the measurements begin a short distance away from the comb in order to avoid any adverse sensing conditions imposed by sensor interactions with the comb itself (e.g., Krowicki 1990). These interactions could generate a concavity or some other aberrant signal at the beginning of the fibrogram. Finally, noise in the sensor can also be a source of concavity.

Moreover, the fibrograms typically produced by the past and current measurement systems, such as the HVI, avoid scanning the beard near the comb. This leaves a gap at the beginning of the resulting fibrogram. However, in order to apply the theory to reconstruct the fiber length distribution, one must have a complete fibrogram. As such, it is desirable to estimate initial missing portion of the fibrogram.

Furthermore, fibrogram data usually includes sensor data beyond the longest fibers in the sample. Theoretically, the value of the fibrogram should be zero after the longest fiber has been scanned. However, in practice, the scanning mechanism of the instrument does not stop at the tip of the longest fiber, and, instead, continues until some fixed length has been reached. Consequently, the signal to noise ratio in this region is low, which can cause issues in processing. As such, since the data in this region has no value, it is useful to determine the approximate length of the longest fibers of the sample and ignore or remove any data beyond that point.

### EXAMPLE 1.3

### Algorithm Details

The algorithm Applicant has developed attempts to resolve the aforementioned practical concerns in two distinct ways. First, the algorithm determines the end of the fibrogram—i.e., the point at which the longest fibers have been scanned after which the remaining data is assumed to be zero. Second, the algorithm reconstructs the initial, missing portion of the fibrogram based on a convex function. Finally, the algorithm applies a windowed curve-fitting procedure, which smooths the curve and removes any slightly concave portions while also providing a way to estimate the derivatives of the fibrogram equation.

### EXAMPLE 1.3.1

### Determining the End of the Fibrogram

The algorithm determines the end of the fibrogram when one of two conditions is met (whichever comes first): (1) the value of the fibrogram is zero, or (2) the first derivative (i.e., first discrete difference) of the fibrogram is greater than or equal to zero.

With respect to the first condition, the fibrogram reaches zero at the length of the longest fiber in the sample. The second condition also comes from the derivation of the fibrogram equation but serves a more practical purpose. In equation 4, one can see that R′(x)<0 for 0<x<Lₘ and R′(Lₘ)=0. In other words, the first derivative is negative over the entire domain except for the longest fiber, Lₘ, at which point it becomes zero. In practice, the region of the curve with the longest fibers becomes noisy and, in some cases, the first derivative (i.e., first discrete difference) may become positive due to noise before the fibrogram reaches zero. In that case. Applicant can conclude the noise is greater than any signal produced by the instrument and, therefore, the data to the right of that point cannot be trusted. As such, all data to the right, x>Lₘ, is simply discarded.

### EXAMPLE 1.3.2

### Estimating the Beginning of the Fibrogram

The initial part of the fibrogram can be estimated by any convex function that behaves similarly to the fibrogram equation 2 in that region of the curve, e.g., 0≤x≤x_{c}, where x_{c} is a cutoff value. The estimating function, g(x), must meet the following conditions: (1) g(x) must be convex for the given interval, and (2) g(x) can be shown to approximate (2) for the given interval.

To estimate the missing part of the fibrogram, the algorithms takes some portion of the left end of the given fibrogram, such as the first 2.54 mm, and use that data to estimate the coefficients of g(x). Then, g(x) is used to extrapolate the curve from the beginning of the given fibrogram back towards the y-axis, i.e. x=0.

In a practical setting, Applicant's observations have revealed that in some cases the first portion of the fibrogram can exhibit some concavity potentially due to sensor interactions with the comb. In those cases, Applicant can use some portion of the given fibrogram to the right of the section that typically exhibits concavity to estimate the coefficients of g(x). Then, using g(x), replace the initial portion of the curve with the fitted curve, g(x), and continue to extrapolate back to the y-axis to fill in the missing portion. For example, suppose one is given a fibrogram that starts at 3.81 mm. The data from 0 mm to 3.81 mm is unknown and must be extrapolated. In addition, consider that the instrument that produced the fibrogram often exhibits concavity in the region from 3.81 to 6.35 mm. In that case, one can estimate g(x) with the data from, e.g., 6.35mm to 12.7 mm and then use g(x) to fill in and replace the fibrogram from 0 mm to 6.35 mm—thereby filling in the missing section from 0 mm to 3.81 mm as well as replacing the potentially concave portion from 3.81 mm to 6.35 mm. Note that this is merely an example of how to apply the general approach described above. Various instruments that produce fibrograms may necessitate different starting points, different ranges, and other parameters, depending on the nature of the instrument and the data it produces, which would require some prior analysis.

Additionally, the option of whether to replace a potentially concave section of the given fibrogram with g(x) can be data-driven on a case-by-case basis. If the initial, known portion of the fibrogram is not concave (which can be measured), then one can fit g(x) to the initial portion of the fibrogram and extrapolate the unknown values. In this case, there is no need to replace the initial, known portion of the fibrogram as in the example above since it is already deemed to be convex.

### EXAMPLE 1.3.3

### Polynomial Smoothing and Extraction of the Distribution

Once the beginning of the fibrogram has been estimated, one can use a sliding window, curve-fitting procedure to smooth the fibrogram to remove any small concavities while simultaneously estimating the underlying fiber length distribution. In practice, Applicant has observed that the middle portion of the fibrogram (for example, 6.35 mm to 25.4 mm) can contain very slight concave sections. Observationally, such concavities within this region typically only occur for a single data point within any given series of points or sometimes two consecutive data points. Different instruments may present different degrees of concavity in this region, but Applicant assumes these anomalies are relatively small.

There are two factors to consider for this part of the algorithm: the size of the window and the type of curve to use in the fitting process. First, the size of the window should largely be determined based on the length and magnitude of the concave regions that tend to occur in the middle part of the fibrogram. The idea is to choose a window size such that the underlying data within the window is generally convex. A window size around 6.35 mm should be sufficient. but this value can be adjusted as needed.

Second, the type of curve to use for the smoothing process must meet the following conditions: (1) the function must be differentiable, (2) the function must be parametric, and (3) when fit to the underlying data in the given window, the function must be convex within the domain of the window.

In addition to the above requirements, as a rule of thumb, the curve should be generally smooth within the window. Additionally, the chosen curve should be capable of providing a good approximation within any window along the fibrogram.

Given a window size and parametric curve, the cumulative distribution function, Y(x), and the probability distribution function, n(x), can be estimated starting with equation 6. As an example, consider a third order polynomial—a function that fits the three criteria above. In that case, R(x) can be approximated by equation 11.

$$R ( x ) \approx {ax}^{3} + b {x}^{2} + c x + d . \qquad ( 11 )$$

Substituting equation 11 along with its derivative with respect to x into equation 6 produces equation 12.

$$Y ( x ) \approx - 2 a {x}^{3} - b {x}^{2} + d . \qquad ( 12 )$$

Taking the derivative of equation 12 with respect to x and substituting into equation 8 produces equation 13.

$$n ( x ) \approx 6 a {x}^{2} + 2 b x . \qquad ( 13 )$$

For a given window size w, let xᵢ be the center of a window whose domain is

$$( {x}_{i} - \frac{w}{2} ) \leq {x}_{i} \leq ( {x}_{i} + \frac{w}{2} ) ,$$

where i=1 . . . n and n is the number of data elements representing the fibrogram. For each xᵢ in the given fibrogram, estimate the coefficients in equation 11 using any appropriate curve fitting method within the given window, e.g., least squares. Y(xᵢ) and n(xᵢ) can then be directly calculated via equations 12 and 13, respectively.

With this sliding window approach, there are special cases that arise at the beginning and end of the fibrogram. At the beginning of the fibrogram where

$$( {x}_{i} - \frac{w}{2} ) < 0 ,$$

part of the window does not contain data. In this case, the coefficients can be estimated with the portion of the window that does contain data, i.e.,

$$0 \leq {x}_{i} \leq ( {x}_{i} + \frac{w}{2} ) .$$

Similarly, the same scenario occurs at the end of the fibrogram when

$$( {x}_{i} + \frac{w}{2} ) > {L}_{m} .$$

Here, again, Applicant can ignore any missing data in the window and simply apply the curve fitting process within

$$( {x}_{i} - \frac{w}{2} ) \leq {x}_{i} \leq {L}_{m} .$$

Regarding these boundary issues, there are many other techniques that can be applied when using sliding windows. Other boundary techniques can be applied with similar results.

### EXAMPLE 1.3.4

### Testing and Validation of the Algorithm

Testing and validation of the algorithm was carried out in two ways. First, Applicant examined the results of applying the algorithm to synthetic distributions. By simulating known distributions, Applicant is able to demonstrate the potential accuracy of the method. Second, Applicant used features derived from the reconstructed length distribution to predict yarn quality of 60 different cotton samples.

### EXAMPLE 1.3.4.1

### Synthetic Distributions

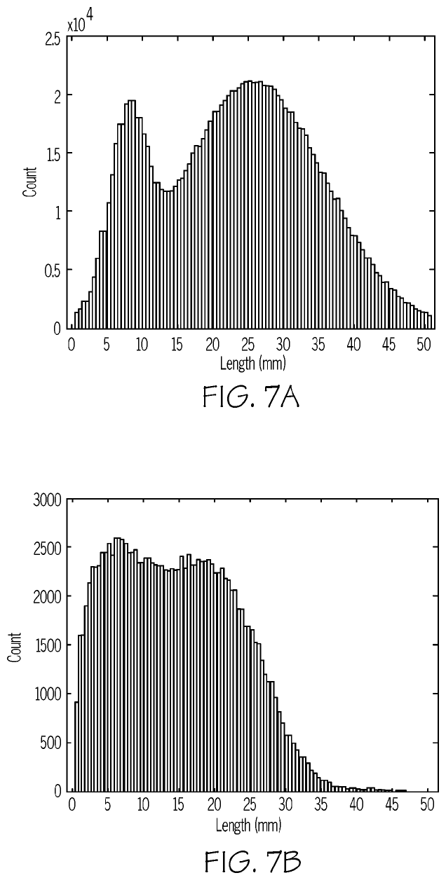

Applicant tested the algorithm on three different synthetic distributions. From each distribution, Applicant sampled one million values representing lengths of cotton fibers. Histograms of these samples are shown in FIGS. 7A-7C. FIG. 7A is a bimodal Gaussian mixture while FIGS. 7B and 7C show Weibull mixtures representing an immature/weak cotton and a mature/strong cotton, respectively, which are taken from Krifa (2008). For each distribution, Applicant calculated the true fibrogram using equation 2 and discretized it to produce 81 data points from 0 mm to 54.61 mm with a spacing of 0.635 mm. Applicant then truncated the data from the fibrogram between 0 mm and 3.81 mm simulating what is provided by a USTER HVI 1000 and then applied the algorithm using the following parameters: Cutoff x_{c}=6.35 mm (i.e., reconstruct the fibrogram from 0 mm to 6.35 mm). Initial part of the curve was reconstructed using the exponential in equation 14.

$$g ( x ) = a {e}^{b x + c} \qquad ( 14 )$$

Equation 14 was estimated using fibrogram data from 6.35 mm to 12.7 mm (window size w=6.985 mm). The curve fitting procedure used a cubic polynomial of the same form as equation 11.

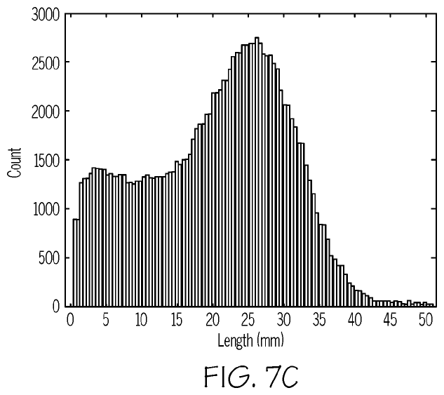

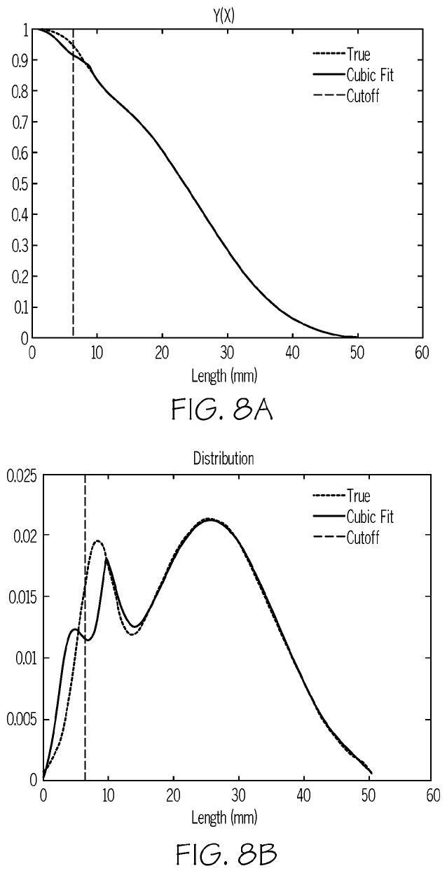

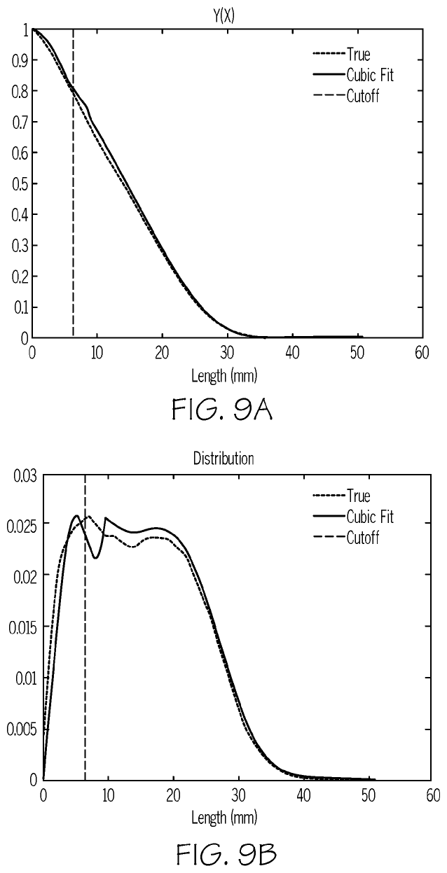

FIGS. 8A-8B, 9A-9B, and 10A-10B show the visual results of estimating Y(x) and n(x) for each of the three synthetic distributions using their fibrograms. In each of these figures, the cutoff location x_{c}=6.35 mm is indicated by a vertical dashed line. FIGS. 8A-8B show the results of the Gaussian mixture model from FIG. 7A. In the reconstructions of both Y(x) in FIG. 8A and n(x) in FIG. 8B, one can see that the greatest error occurs near the cutoff. The reason for this is the slight bump, or lack of smoothness, caused by joining the estimated exponential (equation 14) and the original fibrogram at x_{c}, 6.35 mm. The results of the Weibull mixture distributions shown in FIGS. 9A-9B and 10A-10B exhibit similar behavior. Nevertheless. the reconstructions of each curve show good agreement with the true curves where x>˜12.7 mm.

Along with evaluating observed differences in the graphs, Applicant also examined two of the more commonly used length parameters, mean length (ML) and upper half mean length (UHML). The UHML is computed as the mean length of all fibers longer than the median. These results are shown in Table 1. For each of these parameters, the table shows the true value calculated from the original data drawn from the corresponding distribution and the value calculated from the estimated n(x) as output by the algorithm. As the table shows, the method generally overestimates each value but only by a relatively small margin. For example, the differences for the Gaussian mixture distribution parameters are near 1% or less, while the differences between the more realistic distributions vary between 1.6% and 6.0%.

TABLE 1
Results of computing the ML and UHML for the fibrograms
generated from each type of distribution.

| | ML (mm) | | | UHML (mm) | | |
|---|---|---|---|---|---|---|
| | True | PDF | Error % | True | PDF | Error % |
| Gaussian | 22.71 | 22.95 | 1.1% | 31.89 | 32.08 | 0.6% |
| mixture | | | | | | |
| Immature/ | 14.45 | 15.31 | 6.0% | 21.57 | 22.29 | 3.3% |
| Weak | | | | | | |
| Mature/ | 20.06 | 20.49 | 2.1% | 28.34 | 28.80 | 1.6% |
| Strong | | | | | | |

### EXAMPLE 1.3.4.2

### Yarn Quality Prediction

In addition to testing the algorithm on synthetic distributions, Applicant also validated the algorithm by predicting yarn quality for 60 commercial-like cotton samples. These 60 samples come from a set of Plain Cotton Improvement Committee (PCIC) samples that consist of 12 varieties grown in five counties in the West Texas region in 2016. Each of the samples was tested for fiber properties using HVI and the USTER Advanced Fiber Information System (AFIS). For the HVI, samples were tested for four replications of color/trash, four replications of micronaire, and 10 replications for length and strength. HVI fibrograms were also captured during the measurements of length and strength, and all 10 fibrograms were averaged to provide one fibrogram per sample. For each fibrogram, the length distribution was reconstructed using the proposed algorithm with the same parameters described above for the synthetic distributions (i.e., windows size, cutoff, g(x), etc.). For the AFIS, all samples were tested with three replications. Similarly, the length distributions produced by AFIS were averaged to produce one AFIS length distribution per sample. Each cotton was also ring-spun into 30 Ne yarn (Suessen Elite 1000), and yarn properties were tested (yarn evenness on the USTER Tester 5 and tensile properties on the Statimat DS).

In the following results, Applicant compares yarn quality prediction using the length distribution reconstructed from HVI fibrograms to that of AFIS length distributions. It is important to note that, currently, AFIS is the only high-speed instrument that can produce a fiber length histogram of a cotton sample. It uses an aggressive mechanical opener to separate the fibers and measure them one by one. As such, a chief criticism regarding the AFIS is that it breaks fibers which leads to an overestimation of the number of short fibers in a sample. On the other hand, the HVI measures an entire fiber beard like the one shown in FIG. 2. While the HVI does produce a fibrogram, it only reports two length measurements: UHML and uniformity index (UI), which is the ratio of ML to UHML expressed as a percentage. It is not known how the HVI calculates ML and UHML from the fibrogram, although there is some speculation that they are taken directly from two points along the fibrogram curve (Sayeed, 2020).

In the results presented herein, Applicant compares yarn quality prediction using five standard non-length parameters measured by the HVI: strength, elongation, micronaire, reflectance (Rd), and yellowness (+b). In addition, since fiber length is an important factor in determining yarn quality, Applicant also compared adding UHML and UI derived from three different methods. First, Applicant took these measurements as they are reported from the HVI. Second. Applicant calculated UHML and UI from the AFIS length distribution. Finally, Applicant calculated UHML and UI from the fibrogram-based length distribution output by the algorithm.

Given a model consisting of the five non-length HVI parameters plus UHML and UI taken from one of the three methods, Applicant used partial least squares regression (PLSR) to estimate seven different parameters for yarn quality: tenacity, work-to-break. CVm %. thin places, thick places, neps, and hairiness. Each yarn quality measurement is estimated independently from the others meaning that seven prediction models are generated for each of the three versions of UHML and UI producing a total of 21 predictor-response combinations. Furthermore, leave-one-out cross-validation (LOOCV) is used to evaluate the performance of each model. With this validation method, PLSR is performed 60 times each time leaving out one sample with which to evaluate prediction accuracy—i.e., the difference between the predicted and observed value. For each of the 21 predictor-response combinations, Applicant calculated an average coefficient of determination (R²) among the 60 models generated by LOOCV along with the mean squared error of the test samples left out each iteration.

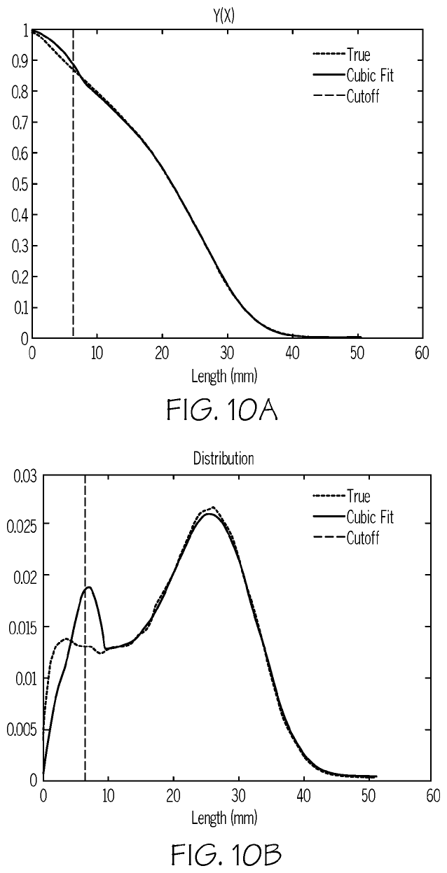

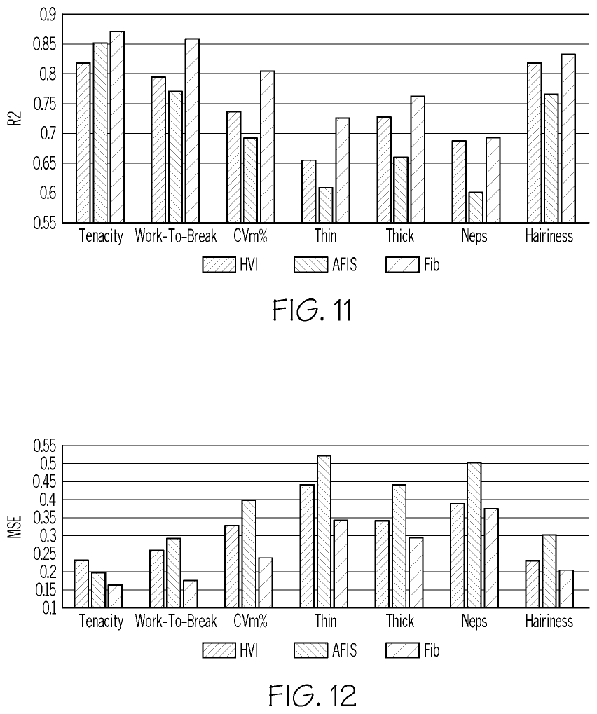

Results for all models are shown in FIGS. 11-12. For each yarn quality parameter. the UHML and UI calculated from the fibrogram-based length distribution (denoted as “Fib” in the legend) perform better than the other two methods. The R² is higher (FIG. 11), and the MSE is lower (FIG. 12).

In sum, Applicant presented a new algorithm for reconstructing a cotton fiber length distribution from a fibrogram. The procedure is based on a sound theoretical framework and uses signal processing techniques to overcome several practical issues that may arise regardless of the sensing method used to acquire the fibrogram signal. Furthermore, the proposed method also allows for flexibility in the selection of some parameters (i.e., windows size, cutoff, g(x), etc.) as fibrograms obtained via other sensing modalities may necessitate slightly different choices. Regardless, the general procedure outlined here would remain the same.

Applicant also presented results that indicate that, not only is the proposed method accurate in its reconstruction based on synthetic distributions. but also provides better length measurements than other leading industry methods when it comes to yarn quality prediction. This method can provide spinning mills (the primary consumers of cotton lint) with an accurate assessment of fiber quality as it directly relates to the quality of spun yarn.

### EXAMPLE 1.4

### Alternative Methods of Determining an End of a Fibrogram

The main part of the algorithm in this Example employs a sliding window, curve fitting procedure that smooths the fibrogram to remove small concavities while simultaneously estimating the underlying fiber length distribution. This process ends when the end of the fibrogram is reached. In this case, the fibrogram has already been truncated.

However, rather than determining the end of the fiber program first, Applicant proposes using the information from the polynomial fit as the algorithm stopping criteria. In theory, the fibrogram reaches zero at the length of the longest fiber and should remain zero for the rest of the signal. Furthermore, the first derivative of the fibrogram is negative over the entire domain except for the longest fiber, at which point the first derivative also becomes zero. However, in practice, this region of the curve is noisy. To overcome this, one can use the result of the polynomial curve fit to determine whether the end of the fibrogram (i.e., the longest fiber) is reached. Using the example provided in the original disclosure, equation 15 considers a 3rd or polynomial as an estimate for the fibrogram curve within a small window around x:

$$R ( x ) \approx a {x}^{3} + b {x}^{2} + c x + d . \qquad ( 15 )$$

The first derivative of R(x) can also be estimated by equation 16.

$${R}^{′} ( x ) \approx 3 a {x}^{2} + 2 b x + c . \qquad ( 16 )$$

With the results of the curve fitting procedure, the coefficients in equation 15 are known, and equation 16 is easily calculated. Using the estimate for R′(x), one could terminate the algorithm when R′(x)=0. However, due to noise in the signal, this precise condition is not likely to occur. Therefore, one must choose some small r and terminate the algorithm when −r≤R′(x)<0. In particular, the end of the fibrogram occurs when R′(x) is sufficiently close to zero. The constant r must be experimentally chosen.

### EXAMPLE 1.5

### References

Chu, Y. T., and Riley, C. R. New Interpretation of the Fibrogram. Textile Res J 1997; 67 (12): 897-901.

Hertel, K. L. A Method of Fiber-Length Analysis Using the Fibrogram, Textile Res J 1940; 10:510-525.

Krifa, M. Fiber Length Distribution in Cotton Processing: A Finite Mixture Distribution Model. Textile Res J 2008; 78 (8): 688-698.

Krowicki, R. S., and Thibodeaux, D. P. Holding Length: Effect on Digital Fibrogram Span Lengths. Textile Res J 1990; 60 (7): 383-388.

Sayeed, A. Improvement of the cotton fiber length measurements using High Volume Instrument (HVI) fibrogram. Texas Tech University. Ph.D. dissertation. 2020.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A computer-implemented method of reconstructing a fiber length distribution of a fiber from a fibrogram of the fiber, said method comprising:
receiving the fibrogram, wherein the fibrogram comprises a curve representing the number of fibers present at a given length from the base of the fiber;
determining an end of the fibrogram;
applying a windowed curve-fitting procedure to smoothen the fibrogram curve; and
estimating the fiber length distribution of the fiber from the smoothened fibrogram curve.

2. The method of claim 1, further comprising a step of reconstructing an initial and missing portion of the fibrogram, wherein the reconstructing occurs through the utilization of a convex function.

3. (canceled)

4. The method of claim 1, further comprising a step of constructing the fibrogram, wherein the fibrogram is constructed through the utilization of a High Volume Instrument (HVI).

5. (canceled)

6. The method of claim 1, wherein the end of the fibrogram represents a length value of the fibrogram that is zero, a first discrete difference of the fibrogram that is greater than or equal to zero, or a point at which the longest fibers have been scanned after which the remaining data is assumed to be zero.

7. (canceled)

8. (canceled)

9. The method of claim 1, wherein a polynomial curve fit procedure is utilized to determine the end of the fibrogram.

10. The method of claim 1, wherein the windowed curve-fitting procedure comprises polynomial smoothing through a sliding window method, wherein the sliding window method utilizes differentiable and parametric functions, and wherein when fit to underlying data in a given window, the function is convex within a domain of the window.

11. (canceled)

12. (canceled)

13. The method of claim 1, wherein the windowed curve-fitting procedure also removes slightly concave portions from the fibrogram curve.

14. The method of claim 1, wherein the windowed curve-fitting procedure also estimates derivatives of fibrogram equations.

15. The method of claim 1, wherein the estimating of the underlying fiber length distribution is simultaneous with the applying of the windowed curve-fitting procedure.

16. The method of claim 1, wherein the estimating of the underlying fiber length distribution comprises estimating a cumulative distribution function and a probability function.

17. The method of claim 1, wherein the estimating of the underlying fiber length distribution is based on a given window size and a parametric curve.

18. The method of claim 17, wherein the window size is selected such that the underlying data within the window is generally convex.

19. The method of claim 17, wherein the curve for the smoothing process has a function that is differentiable and parametric, and when fit to the underlying data in the window, the function is convex within the domain of the window.

20. The method of claim 17, wherein the curve is generally smooth within the window.

21. The method of claim 17, wherein the curve is capable of providing a good approximation within any window along the fibrogram.

22. The method of claim 1, wherein the fiber is in the form of an aggregated fiber, and wherein the aggregated fiber is in the form of a fiber beard, a fiber bundle, a yarn, or combinations thereof.

23. (canceled)

24. (canceled)

25. The method of claim 1, wherein the fiber is selected from the group consisting of textile fibers, cotton fibers, hemp fibers, natural bast fibers, flax fibers, jute fibers, kenaf fibers, milkweed fibers, ramie fibers, artificial fibers, or combinations thereof.

26. (canceled)

27. (canceled)

28. The method of claim 1, further comprising a step of assessing fiber quality based on the estimated fiber length distribution.

29. The method of claim 1, further comprising a step of adjusting one or more fiber-related conditions based on the estimated fiber length distribution.

30. The method of claim 29, wherein the adjusting comprises instructing a user to adjust the one or more fiber-related conditions based on the estimated fiber length distribution.

31. The method of claim 29, wherein the adjusting occurs manually by a user.

32. The method of claim 29, wherein the one or more fiber-related conditions are selected from the group consisting of fiber growth conditions, fiber storage conditions, fiber milling conditions, fiber transport conditions, fiber breeding conditions, or combinations thereof.

33. (canceled)

34. A computing device for reconstructing a fiber length distribution of a fiber from a fibrogram of the fiber, wherein the computing device comprises one or more computer readable storage mediums having a program code embodied therewith, wherein the program code comprises programming instructions for:
receiving the fibrogram, wherein the fibrogram comprises a curve representing the number of fibers present at a given length from the base of the fiber;
determining an end of the fibrogram;
applying a windowed curve-fitting procedure to smoothen the fibrogram curve; and
estimating the fiber length distribution of the fiber from the smoothened fibrogram curve.

35. The computing device of claim 34, wherein the program code further comprises programming instructions for reconstructing an initial and missing portion of the fibrogram.

36. The computing device of claim 34, wherein the program code utilizes a polynomial curve fit procedure to determine the end of the fibrogram.

37. The computing device of claim 34, wherein the programming instructions for the windowed curve-fitting procedure comprises polynomial smoothing through a sliding window method, wherein the sliding window method utilizes differentiable and parametric functions, and wherein when fit to underlying data in a given window, the function is convex within a domain of the window.

38. (canceled)

39. The computing device of claim 34, wherein the programming instructions for the windowed curve-fitting procedure also removes slightly concave portions from the fibrogram curve.

40. The computing device of claim 34, wherein the programming instructions for the windowed curve-fitting procedure also estimates derivatives of fibrogram equations.

41. The computing device of claim 34, wherein the programming instructions for estimating of the underlying fiber length distribution is simultaneous with programming instructions for the applying of the windowed curve-fitting procedure.

42. The computing device of claim 34, wherein the programming instructions for estimating of the underlying fiber length distribution comprises instructions for estimating a cumulative distribution function and a probability function.

43. The computing device of claim 34, wherein the programming instructions for estimating of the underlying fiber length distribution is based on a given window size and a parametric curve, wherein the window size is selected such that the underlying data within the window is generally convex, wherein the curve for the smoothing process has a function that is differentiable and parametric, and when fit to the underlying data in the window, the function is convex within the domain of the window.

44. The computing device of claim 43, wherein the curve is generally smooth within the window.

45. The computing device of claim 43, wherein the curve is capable of providing a good approximation within any window along the fibrogram.

46. The computing device of claim 34, wherein the program code further comprises programming instructions for assessing fiber quality based on the estimated fiber length distribution.

47. The computing device of claim 34, wherein the program code further comprises programming instructions for instructing the adjustment of one or more fiber-related conditions based on the estimated fiber length distribution, wherein the one or more fiber-related conditions are selected from the group consisting of fiber growth conditions, fiber storage conditions, fiber milling conditions, fiber transport conditions, fiber breeding conditions, or combinations thereof.

48. (canceled)

49. (canceled)
